(19) United States
(12) Patent Application Publication
(10) Pub. No.: US 2009/0210322 A1
(43) Pub. Date: Aug. 20, 2009

(54) METHOD FOR CREATING CUSTOM BLENDED COSMETICS

(76) Inventor: Karen Elizabeth Gould Stark, Trumansburg, NY (US)

Correspondence Address: BROWN & MICHAELS, PC;400 M & T BANK BUILDING, 118 NORTH TIOGA ST, ITHACA, NY, 14850, US

(21) Appl. No.: 12/369,812

(22) Filed: Feb. 12, 2009

Related U.S. Application Data
(60) Provisional application No. 61/029,014, filed on Feb. 15, 2008.

(51) Int. Cl. G06Q 30/00; G06Q 50/00; A61K 8/00; A61Q 1/02

(52) U.S. Cl. 705/27; 424/63

(57) ABSTRACT

A method for custom blending eye shadow, facial and lip cosmetics by a consumer. The consumer may purchase a cosmetic color kit that contains one or more base powders, along with a measuring device, a container for mixing the ingredients, and instructions for custom blending essentially an infinite variety of final cosmetic colors, or they may purchase individual components of the kit. The advantage is that the consumer can modify the custom blended cosmetic, if their first, second or even third attempt at making the exact color desired has not yet been achieved. They need not purchase a variety of single colors in an attempt to find the right one for their cosmetic needs.

### REFERENCE TO RELATED APPLICATIONS

This application claims one or more inventions which were disclosed in Provisional Application No. 61/029,014, filed Feb. 15, 2008, entitled “Method for Creating and Selling Custom Blended Cosmetics”. The benefit under 35 USC §119(e) of the United States provisional application is hereby claimed, and the aforementioned application is hereby incorporated herein by reference.

### FIELD OF THE INVENTION

The invention pertains to the field of cosmetics. More particularly, the invention pertains to a method for consumer blended facial, eye shadow and lip cosmetics.

### BACKGROUND OF THE INVENTION

Custom blended foundations are available in some department stores and spas, however these products are typically liquid type products and there still remain issues with lighting, and shade shifting, thereby making it difficult to ensure that the color match is accurate. These factors lead to dissatisfied customers, product returns and lost repeat business. Because of sanitary issues, products that are returned generally cannot be resold, and this creates financial losses for retailers.

The process of finding a good color match is sometimes difficult, time consuming, and costly. Retailers must stock numerous foundation colors, and the consumer must then try to pick a shade that is ideal for them from a wide range of products. It is not unusual for someone to try or purchase several different shades to find a good match. It is also not unusual for someone to purchase a foundation thinking it that it is a good color match-only to go into a different lighting situation and find that the product they purchased is not the right color for them. Typical foundations also blend with a person's unique body chemistry which can alter the color throughout the course of a day. Even if someone does find a good match, such as if they get a tan or their tan fades, then their foundation will cease to match and the process of finding an appropriate shade match starts again.

Foundation was invented in the 1920's by Max Factor to give women a way to hide flaws, and create the appearance of a smooth, even skin tone. Max Factor's foundations consisted of ingredients to provide formulations—generally consisting of titanium dioxide and or zinc oxide, which were then tinted with various pigments to allow for the matching of skin tones. Until the 70's most foundations were sold as a liquid or a paste-like stick. In the 1970's some companies started to sell “mineral” foundation, that is, those that generally utilized standard cosmetic ingredients like titanium dioxide and iron oxides—blended with a powder type filler rather than the traditional emulsion based foundations.

### SUMMARY OF THE INVENTION

The present invention consists of a method of custom blending facial, eye shadow and lip cosmetics by the consumer. The consumer purchases individual components or a cosmetic color kit that contains one or more base powders, along with a measuring device, a container for mixing the ingredients, a variety of colored powder additives and instructions for custom blending essentially an infinite variety of final cosmetic colors. The advantage is that the consumer can modify the custom blended cosmetic, if their first, second or even third attempt at making the exact color desired has not yet been achieved.

The mixing media for powdered facial cosmetics consists of powdered “fillers” including by not limited to mica, sericite, bismuth oxychloride, boron nitride, kaolin, talc, or powdered starches, plus ingredients for oil control, adhesion coverage and esthetics, as necessary. The mixing medium for lip products is an anhydrous carrier medium including but not limited to one or more of the following: liquid oils, liquid waxes, semi-solid fats and oils, and solid waxes.

The base powders come in a variety of standard colors, such as, for example white, yellow, ivory, brown, ebony and peach. The colors of the base powder are not limited to these examples and may include other colors. The addition of varying amounts of the base powders provides the consumer with a virtually endless spectrum of cosmetic colors.

### BRIEF DESCRIPTION OF THE DRAWING

FIG. 1 shows a flow chart for custom blending a desired cosmetic according to the method of this invention.

### DETAILED DESCRIPTION OF THE INVENTION

In the practice of this invention the potential consumer visits an online or retail establishment. In a retail establishment, a clerk or makeup artist could blend the colors for the consumer, or there may be a “self service” counter where consumers can blend their own colors with instructions from a clerk. They may purchase a customized starter kit that includes one or more mixing mediums, empty containers, a measuring spoon, colored bases, and a sheet that includes instructions and “recipes” for mixing an almost infinite variety of colors. The mixing spoon is approximately 1/32 teaspoon, but larger ones could also be included. The mixing mediums for facial foundation, concealers, and eye shadows are generally composed of a blend of some or all of the following ingredients: powdered “filler”, such as sericite and other micas, titanium dioxide, zinc oxide, magnesium or zinc stearate and sebum control ingredients like silica, kaolin, powdered starches, or silk powder. The mixing medium for blushes would primarily consist of translucent powdered “filler” such as sericite, mica, silk powder, or vegetable starch. The colored bases come in a variety of standard colors, such as, for example white, yellow, brown, ebony and peach. However, the scope of potential colors of the base powder is not limited to these examples and may include other colors as well.

Preferred base compound formulations include the following ranges of ingredients:

30-70% filler

5-75% titanium dioxide

5-75% zinc oxide

Remainder, sebum control, adhesion and estethic ingredients.

Colored bases also include between 10% and 60% tinting pigments added to the above compound, or colorless filler. Ideal tinting pigments include but are not limited to iron oxides, ultramarines, synthetic and natural dyes.

Eye shadow bases are essentially the same as the ingredients used for facial cosmetics. Only different proportions would be used and are covered by the preferred base compound shown above.

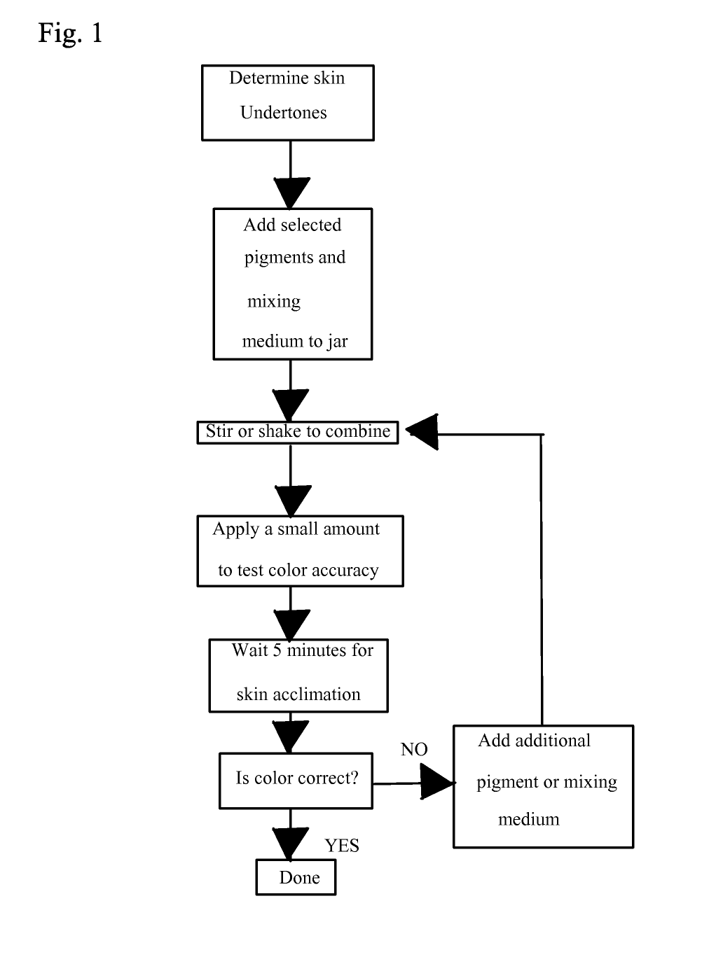

For an example of a facial cosmetic for someone with a pale Caucasian skin tone, one part white base to 12 parts neutral brown “ebony” base may be suitable. Referring to FIG. 1, a person first determines their skin undertones 10, then adds selected base pigments to a mixing container 14 and shake or stir the ingredients 16. In this manner they can custom blend a perfect color match, repeat the results when they run out, or easily adjust their color if their skin color changes or they decide that the recipe they used wasn't accurate or otherwise suitable. Other examples of common blends include the following. For a person having light to medium skin, one would start with approximately 5 to 6 scoops of white and one scoop of ebony. For a person having medium to tan skin, one would mix one scoop of white and one scoop of ebony. For a person having very light skin color, approximately 12 scoops of ivory and 1 scoop of ebony might be appropriate. Of course, after initial blending, the user would test a small area of the blend either on their cheek, jaw-line or forehead 16, then let the cosmetic sit for a few minutes to blend with the user's natural tone and oils 18 to determine if the blended color is as desired 20. Further adjusting of the colors may then be necessary by adding one or more scoops of the appropriate base color 22.

Darker blends may be tinted with a blend of iron oxides and other pigments using high shear mixing to disperse and pulverize the pigments. The tinted bases may contain up to approximately 75% pigment. The customer can then combine those powders to create a close match to their own natural skin tone. If the customer's skin tone changes, or they find that the mixture that they just blended is unsatisfactory, they can easily adjust their blend by adding more of one of the bases to the existing mixing container until the desired color is achieved.

Lip product mixing mediums may contain liquid oils, triglycerides, plant butters, lanolin or other animal fats, hydrogenated oils, petroleum or silicone based products, waxes, oil soluble vitamins, anti-oxidants to prevent rancidity, functional or conditioning agents and flavorings.

The method and kit of the present tinting pigments are pre-dispersed in a powder base. This permits the customization of color without the use of additional equipment like a blender or grinder. Only shaking or stirring is required. This significantly simplifies the process and provides the customer with infinitely more options than with purchasing a single or a variety of separate colors at a retail establishment.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method for acquiring custom blending cosmetics from either a supplier website or a retail outlet comprising the steps of:
a) a potential consumer reviewing a plurality of blending components that may be purchased, and
b) the potential consumer purchasing one or more of the plurality of blending components.

2. The method of claim 1 wherein the custom blended cosmetic is selected from the group consisting of eye shadow, lip and facial cosmetics.

3. The method of claim 1 wherein of custom blending components are purchased as a kit containing one or more base powders, a measuring spoon, a container for mixing, and an instruction sheet with various “recipes” for mixing an almost infinite variety of colors.

4. The method of claim 1 wherein the one or more base powders are selected from the group consisting of white, yellow, brown, ebony and peach.

5. A method for custom blending cosmetics by a consumer comprising the steps of:
a) determining which color the consumer desires,
b) measuring an amount of a base powder and depositing it into a container,
c) measuring an amount of one or more of a plurality of colored bases and depositing that amount into the container, and
d) mixing the ingredients from (b) and (c) in the container.

6. The method of claim 5 where the custom blended cosmetics are selected from the group of eye shadows, facial and lip cosmetics.

7. The method of claim 5 where the plurality of colored bases are selected from the group consisting of white, yellow, ivory, brown, ebony and peach.

8. The method of claim 5 where the custom blended cosmetics are in a powder form.

9. The method of claim 5 further comprising adding more of a colored base to achieve the exact desired color if the precise color desired is not achieve from step (d) of claim 5.

10. The method of claim 5 where mixing is achieved either stirring or shaking the container.

11. The method of claim 5 where the consumer may be the end user or an employee of a spa or retail establishment

11. A kit for custom blending cosmetics by a consumer comprising:
a) at least one colored base,
b) a measuring spoon,
c) a container for mixing, and
d) an instruction sheet having a plurality of recipes for mixing an essentially endless spectrum of colors.

10. The kit of claim 9 wherein the at least one colored base is selected from the group consisting of white, yellow, ivory, ebony, brown and peach.

11. The kit of claim 9 wherein the measuring spoon is approximately 1/32 of a teaspoon.
